(19) United States
(12) Patent Application Publication
(10) Pub. No.: US 2004/0106899 A1
(43) Pub. Date: Jun. 3, 2004

(54) Gastric balloon catheter with improved balloon orientation

(76) Inventors: Donald J. McMichael, South Jordan, UT (US); Michael Allen Kenowski, Pocatello, ID (US); Thomas Gregory Triebes, Alpharetta, GA (US); Netty Dawn DiViesti, Pocatello, ID (US)

Correspondence Address: KIMBERLY-CLARK WORLDWIDE, INC., 401 NORTH LAKE STREET, NEENAH, WI, 54956

(21) Appl. No.: 10/306,993

(22) Filed: Nov. 30, 2002

(51) Int. Cl. A61M 29/00

(52) U.S. Cl. 604/104; 604/910; 604/96.01

(57) ABSTRACT

A balloon catheter with improved balloon orientation includes a head, a catheter shaft extending from the head, and a balloon disposed on the catheter shaft opposite the head. The balloon of the present invention has an orientation such that the inflated width of the balloon is at least about 1.3 times the length of the balloon.

### BACKGROUND

Catheterization of a body cavity is frequently performed in medical procedures either to insert substances into or to remove substances from the body. During many of these procedures, it is necessary to keep the catheter in a relatively stable position to perform the desired insertion or removal. With the use of enteral feeding catheters (i.e., catheters which enable the administration of nutritional solutions directly into the stomach or intestines), for example, it is necessary to ensure that the catheter is not accidentally removed from the stomach or intestines. This is true both during the actual administration or removal of fluids, and the time periods in between.

In order to ensure that a catheter is maintained in the proper position, it is common to use a balloon disposed near the distal (patient) end of the catheter shaft. Inflating the balloon causes the balloon to contact the anatomical structure (i.e., a duct or stomach wall) and thereby prevents the catheter from moving out of the proper position. In the case of enteral feeding, a stoma is formed leading into the stomach or intestine. The catheter is positioned to extend through the stoma so as to form a channel into the stomach or intestines through which enteral feeding solutions may be instilled.

FIG. 1 shows a side view of a prior art balloon catheter 10 having a head 14 disposed at a proximal end 15 of the catheter 10. The head 14 contains valves (not shown) which regulate the flow of fluids through the balloon catheter 10. The head 14 also prevents the balloon catheter 10 from completely advancing through the stoma and into the stomach or intestine of the user.

To prevent the catheter 10 from being pulled out of the stomach/intestinal wall, a balloon 18 is disposed along a catheter shaft 26. The catheter 10 is shown having an optional stiff tip 30, which is attached to the catheter shaft 26 at a distal end 17 opposite the head 14. The catheter shaft 26 is typically made of a medical grade silicone. The stiff tip 30, when present, is also frequently formed of a medical grade silicone but is usually configured to be as rigid as or less rigid than the catheter shaft 26 through the stoma.

The balloon 18 has a balloon proximal end 20 attached to the catheter shaft 26 by the use of adhesive, thereby forming a proximal cuff 32. Likewise, the balloon distal end 22 is adhesively attached to the catheter shaft 26 and/or stiff tip 30, thereby forming a distal cuff 34.

The balloon 18 is advantageous because it allows the catheter shaft 26 to be inserted into the stoma (not shown) while the balloon 18 is uninflated. Once the catheter shaft 26 is properly positioned in the stoma, a syringe (not shown) is inserted into a side port 36 of the head 14 and a fluid is injected into the balloon 18 through a lumen (not shown in FIG. 1) of the catheter 10 so as to inflate the balloon 18.

While the balloon 18 remains inflated, the catheter 10 stays properly positioned in the stoma. The position of the balloon catheter 10 is maintained in such a manner until removal is desired. If the catheter 10 needs to be removed, the balloon 18 may be deflated so that it will not interfere with withdrawal of the catheter shaft 26 and stiff tip 30.

The type of balloon 18 shown in FIG. 1 is fashioned around the perimeter of the catheter shaft 26 such that when it is deflated it reduces or contracts about the shaft 26 but is still clearly larger than overall diameter of the catheter.

Attachment of the balloon 18 to the catheter shaft 26 is frequently accomplished by gluing the balloon proximal end 20 and the balloon distal end 22 to corresponding positions on the external surface of the catheter shaft 26 so as to form a proximal cuff 32 and a distal cuff 34, respectively. Such cuffs 32 and 34 are longitudinal sections of the balloon 18 whose inside diameters correspond to the outside diameter of the shaft 26 at their respective points of attachment to the catheter 10 and have a distance between them which is about the length of the uninflated balloon 18. The cuffs 32 and 34 must be of sufficient length to provide a tight and durable seal between the balloon 18 and the catheter shaft 26.

FIG. 2 shows a side view of another prior art balloon catheter 110. The catheter 110 is generally similar to catheter 10 (FIG. 1) except that the head 114 (FIG. 2) of catheter 110 is a large or non-low profile head and is adapted to extend well beyond the patient's body. While the balloon 18 of catheter 10 may be located at or near the distal end 17 of catheter shaft 26, as shown in FIG. 1, FIG. 2 also shows that balloon 118 may be located more inwardly of the distal end 117 of the catheter 110 (i.e. more proximal to the head 114).

While the prior art balloon configurations shown in FIGS. 1 and 2 work to maintain the balloon catheters 10 and 110, respectively, in the proper position within the patient, those balloon catheters as well as the other known balloon catheters do have disadvantages. For example, some embodiments of prior balloon catheters have a small surface area of the balloon contacting the patient, a leaky seal, a concentration of pressure or force against the patient over a small area, and/or constriction of the feeding lumen as a result of the pressure applied to the catheter by the inflated balloon.

Another disadvantage is discomfort to the user. For example, in either of the two prior art catheters illustrated, but especially with regard to the catheter of FIG. 1, in order to allow insertion of the catheter 10, the catheter shaft 26 and the stiff tip 30 (when present) must be relatively rigid or firm to prevent buckling under insertion pressures. However, this same firmness makes the distal tip 30 much more prone to irritate anatomical structures which come into contact with it. This is especially true in the stomach and intestines where the opposing walls of the anatomical structures tend to collapse on each other during physical exertion or when the cavity has little or no food. As the person moves, the stiff tip 30 repeatedly engages the adjacent anatomical structure (such as the stomach wall) and can lead to irritation and/or discomfort for the user. Thus, as the presence of an extended stiff catheter tip in this environment has been suspected of irritating the opposing surfaces of the body cavity, it would be desirable if the patient could be protected from exposure to such a tip 30.

Accordingly, there is a need in the art for a balloon catheter with a stiff distal tip isolated from opposing internal body cavity surfaces.

Another disadvantage experienced with some of the current catheters, especially when they are used on smaller patients (e.g. neonates or small children) or other individuals having small stomachs, is that the balloon takes up a significant portion of the stomach. As such, the amount and/or duration of a feeding may be limited due to the availability of stomach space which is not filled by the balloon.

Thus, there is a need for an improved balloon catheter having a balloon which has a low profile orientation and which may also have a low volume relative to traditional balloon catheter balloons.

### SUMMARY OF THE INVENTION

In response to the difficulties and problems discussed above, a new balloon catheter with improved balloon orientation and dimensions has been developed. One aspect of the present invention relates to a catheter having an interior and an exterior, a shaft and a means for expansion. More specifically, the catheter includes a means for expansion having a proximal end, a distal end, a length dimension and a width dimension; a shaft having a distal end, a first lumen configured for fluid communication with a body cavity, and a second lumen configured for communication with the means for expansion. The catheter is designed such that the width dimension of the means for expansion is greater than the length of the means for expansion when the means for expansion is expanded.

Another aspect of the present invention relates to a catheter having a head, a catheter shaft and a means for expansion. More specifically, the head of the catheter has at least two openings through which fluid may be passed. The catheter shaft has an a first and second lumen, each lumen being in communication with at least one of the two openings. The catheter shaft has an interior and an exterior and generally extends from the head. The means for expansion of the catheter has a first end attached to the catheter shaft and a second end attached to the catheter shaft, such that the means for expansion has a width at least about 1.4 times greater than the length of the means for expansion when the means for expansion is expanded.

Another aspect of the invention relates to a catheter including an expandable balloon and an elongate shaft having at least one lumen extending longitudinally therethrough, a distal end and a width dimension. The catheter is designed such that the width of the balloon is larger than the length when the balloon is expanded.

Yet another aspect of the present invention is directed to a balloon catheter configured for placement through a stoma into a body cavity so that the catheter is maintained in the stoma. The catheter includes a head having at least one opening through which a fluid may be introduced; a catheter shaft extending from the head portion to a distal tip, the catheter shaft extending from the head to a distal end, the shaft forming a passageway having an interior; and an expandable member attached to the catheter shaft about the passageway so as to form a balloon. The balloon is such that when expanded the balloon extends radially outwardly from the catheter shaft such that the width of the balloon is at least about 1.3 times the length of the balloon.

These and other features and advantages will be seen from the following detailed description of the drawings and from the claims.

### BRIEF DESCRIPTION OF THE DRAWINGS

The above and other objects, features and advantages of the invention will become apparent from a consideration of the subsequent detailed description presented in connection with the accompanying drawings in which:

FIG. 1 shows a side view of a prior art balloon catheter;

FIG. 2 shows a side view of a second prior art balloon catheter;

FIG. 3 shows a side view of a balloon catheter made in accordance with the teachings of the present invention;

FIG. 4 shows a cross-sectional view of the balloon catheter of FIG. 3;

FIG. 4A shows an enlargement of the encircled area of FIG. 4;

FIG. 4B shows an alternate embodiment of a portion of the encircled area of FIG. 4;

FIG. 5 shows the balloon catheter of FIG. 3 with the balloon in an inflated configuration;

FIG. 6 shows a cross-sectional view of the balloon catheter of FIG. 5;

FIG. 6A shows an enlargement of the encircled area of FIG. 6; and

FIG. 7 shows a side view of another balloon catheter made in accordance with the teachings of the present invention.

### DETAILED DESCRIPTION OF THE PRESENT INVENTION

Reference will now be made to the drawings in which the various elements of the present invention will be given numeral designations and in which the invention will be discussed so as to enable one skilled in the art to make and use the invention. It should be appreciated that each example is provided by way of explaining the invention, and not as a limitation of the invention. For example, features illustrated or described with respect to one embodiment may be used with another embodiment to yield still a further embodiment. These and other modifications and variations are within the scope and spirit of the invention.

It will be appreciated that while reference is made to a means for expansion, means for expansion may also mean or include, but is not limited to, a balloon, a sleeve, an inflatable or expandable member, an elastomeric sleeve, an expandable region or portion, an inflatable member, any other suitable expansion means or the like. However, for ease of reading and understanding of this disclosure, and not intending to be limited thereby, means for expansion will hereinafter be referred to as a balloon. It will also be appreciated that throughout the disclosure reference is made to inflation of the balloon, however, the present invention is not intended to be limited only to inflation. That is, while inflation is used herein for purposes of ease of reading and understanding the disclosure, the term inflation is also intended to mean or include, but is not limited to, expansion, enlargement, swelling or the like.

It will be also appreciated that while reference is made to a tip member, the term tip member is contemplated to mean or include, but is not limited to, tips of all shapes and sizes, a tip region, tip portion, a unitary component, the portion of a unitary component containing a tip member, or the like. However, the term tip member will used throughout the remainder of the disclosure in place of the other terms for ease of reading and understanding the disclosure.

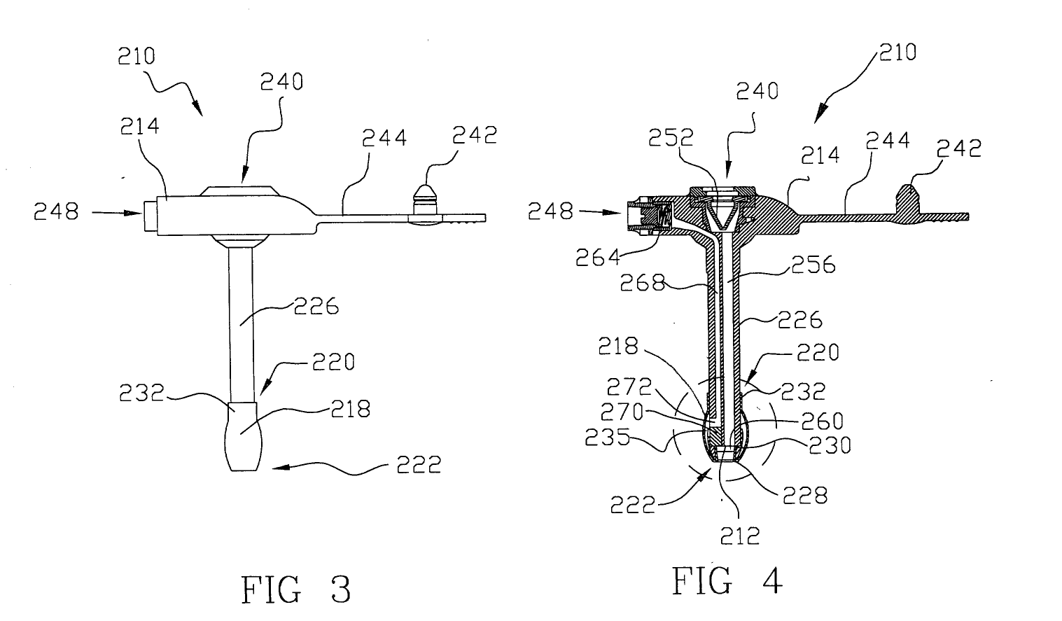

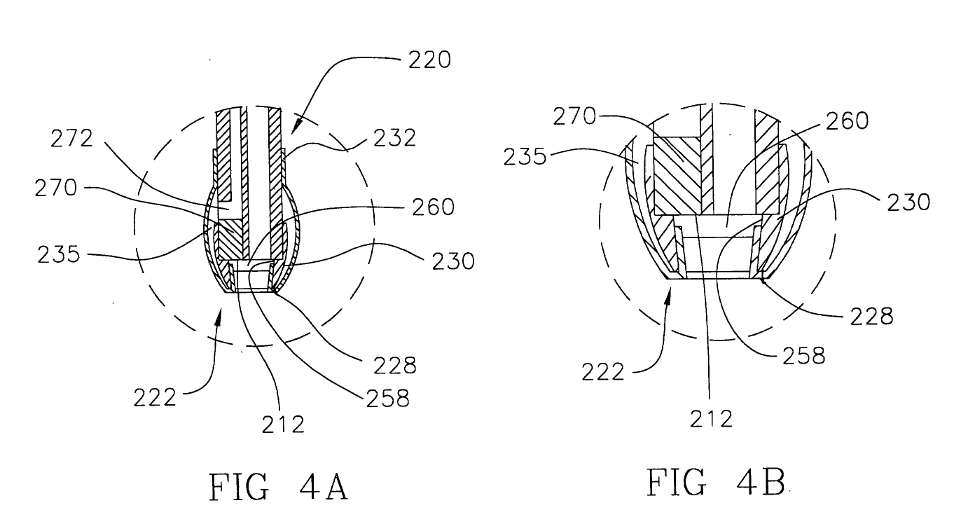

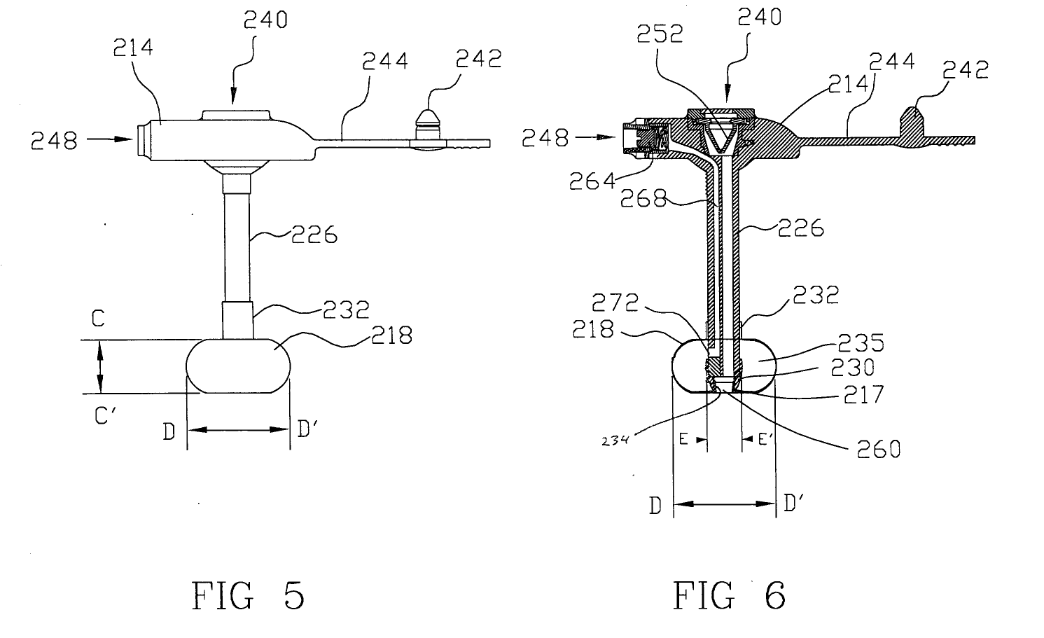

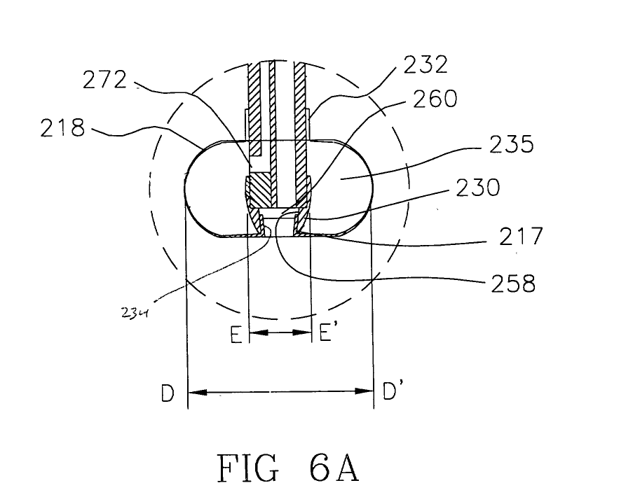

Referring now to FIGS. 3-6A, there is shown a side view and a cross-sectional view, respectively, of a catheter 210 made in accordance with the teachings of the present invention. The catheter 210 includes a proximal head 214 (FIGS. 3, 4, 5, and 6), a shaft 226 and a balloon 218 (FIGS. 3-6A). The catheter shaft 226 (FIGS. 3, 4, 5, and 6) has a distal end 212 (FIG. 4), a first lumen 256 (FIGS. 4 and 6) adapted for fluid communication with a body cavity (not shown), and a second lumen 268 (FIGS. 4 and 6) adapted for communication with the balloon 218. The catheter 210 (FIGS. 4, 4A, and 6) also shows an optional stiff tip 230 (FIGS. 4, 6 and 6A) attached at the distal end 212 (FIG. 4) of the catheter shaft 226. The stiff tip 230 (FIGS. 4, 4A, 4B and 6A) has an interior wall or surface 258 (FIGS. 4A and 4B) which defines a passageway 260 (FIGS. 6 and 6A) which is configured for the passage of fluids therethrough and into or out of the catheter 210. The balloon 218 has a proximal end 220 (FIGS. 3, 4 and 4A), a distal end 222 (FIGS. 3, 4, 4A and 4B), a length dimension and a width dimension. The width dimension, as viewed in a left-to-right direction in FIG. 5, of the balloon 218 is greater than the length dimension, as viewed in a top-to-bottom direction, of the balloon when inflated. As illustrated in FIG. 5, the width dimension is indicated by arrows D and D′ while the length dimension of the balloon is indicated by arrows C and C′.

A first or proximal opening 240 (FIGS. 3, 4, 5 and 6) in the head 214 (FIGS. 3, 4, 5, and 6) allows for the introduction of fluid, bolus feeding or the provision of other nutrient fluids, formula, or the like through the catheter shaft 226, more specifically lumen 256 and a passageway 260 through the stiff tip 230 attached to the distal end 212 of the catheter shaft 226, and into the user. Although not required, an anti-reflux valve 252 (FIGS. 4 and 6), which is generally included to prevent back-flow of the fluids unless properly engaged, is shown disposed between the opening 240 and the first lumen 256.

Inflation or second opening or port 248 (FIGS. 3, 4, 5 and 6) is disposed in head 214 and communicates with the inflation lumen 268 (FIGS. 4 and 6) which extends longitudinally through the shaft 226. The inflation lumen 268 is shown as terminating laterally to the shaft 226 at port 272 (FIGS. 4, 4A, 6 and 6A) into the cavity 235 (FIGS. 4A, 4B and 6) created by the balloon 218 and the shaft 226, as discussed in more detail below. A one-way valve 264 (FIGS. 4 and 6) may be desirably disposed between the inflation port 248 and the inflation lumen 268. Application of positive fluid pressure, such as with air or saline, within and/or upon the inflation lumen 268 by way of the inflation port 248 may cause the balloon 218 to inflate. One-way valve 264, inflation port 248, and inflation lumen 268 are in control of or in communication with the balloon 218 such that a user or clinician may selectively control inflation and deflation of the balloon 218. Valve 264 helps prevent inadvertent deflation of the balloon 218. Also shown associated with the head 214 is a plug 242 adapted for the proximal opening 240 and a lanyard 244 for retaining the plug 242.

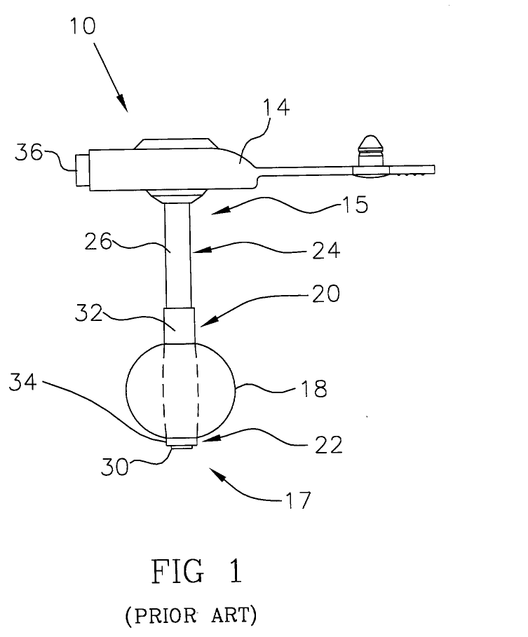

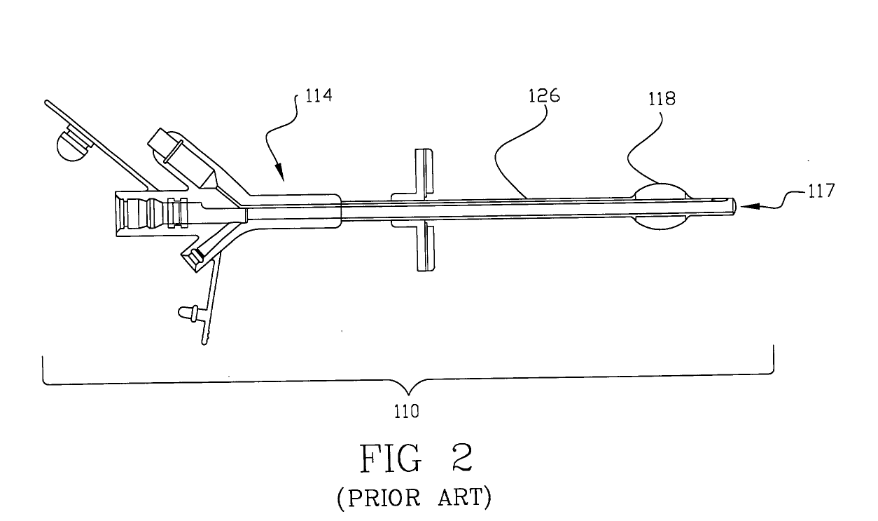

The balloon 218 (FIGS. 3, 4, 5, 6, and 6A) may be attached along the catheter shaft 226 so as to be coaxial therewith. That is, the balloon proximal end 220 (FIGS. 3, 4 and 4A) may be attached so as to form a proximal cuff 232 (FIGS. 4, 4A, 5, and 6). The distal end 222 (FIGS. 3-6) of the balloon 218 may also be secured to the shaft 226. The distal end 222 of the balloon 218 may be secured to the catheter 210 in any number of suitable manners, thereby resulting, for example, in a distal cuff 234 (FIGS. 4, 4A, 4B and 6). However, unlike the prior art balloon catheter 10 of FIG. 1, the distal end 222 (FIGS. 3-6) of the balloon 218 need not be attached several millimeters from the distal end 228 (FIGS. 4, 4A and 4B) of the tip 230 (FIGS. 4, 4A, 4B, 6 and 6) along the exterior surface of the tip 230. Rather, in some embodiments (e.g., FIGS. 4A, 4B, 6 and 6A) of the present invention the balloon distal end 222 may be disposed in such a manner that the balloon 218 covers the stiff tip 230, thereby preventing or reducing the frequency of contact between the stiff tip 230 and anatomical structures adjacent thereto. More specifically, some embodiments of the present invention provide for the distal end 222 of the balloon 218 to be wrapped over the distal end 228 of the tip 230, and attached to an interior surface or wall 258 of the tip 230. In such an embodiment, the distal end 222 of the balloon 218 will cover the distal end 228 of the stiff tip 230 such that the balloon 218 will prevent the stiff tip 230 from directly impacting anatomical structures disposed adjacent thereto when the balloon is inflated thereby minimizing the irritation between the tip 230 of the catheter 210 and the adjacent tissue in a patient associated with prior art gastric balloon catheters.

It will be appreciated that the size of the catheter 210 as well as the length (inflated and uninflated) of the balloon 218 may be varied in accordance with the size and shape of the body cavity (not shown) the catheter 210 is to be used in and the nature of the matter to be moved through the catheter 210. That is, in some instances, it may be desirable to use catheters 210 having larger and/or wider shafts 226 than in other embodiments. Additionally, as discussed in more detail below the balloon 218 of the catheter 210 may be designed to have a certain size and/or shape in either or both of its inflated or uninflated configurations.

It will be appreciated that the various components of the catheters 210 may be made of any suitable material and may desirably be formed from bio-compatible materials such as medical grade silicone or the like. While valves 252 and 264 may be formed of any suitable material they are desirably made of a suitable polymer such as polycarbonate.

It will be further appreciated that the length of the balloon 218 as well as the point along the shaft 226 at which the end 220 of the balloon 218 is attached may effect the shape of the resulting balloon 218. Another suitable way of controlling the shape of the resulting inflated balloon 218 includes annular rings (not shown). Another way of controlling the shape of the inflatable balloon includes, but is not limited to, rotational dipping, commonly done in the condom industry in order to create a uniform film. Still other suitable ways of controlling the shape of the resulting balloon may include, but are not limited to, those discussed in U.S. Pat. No. 6,264,631 B1 to Willis et al., which is incorporated by reference in its entirety.

As noted above, the balloon 218 may be attached to the catheter 210 in a variety of manners as well as a variety of locations. For example, the attachment of balloon 218 may desirably be achieved by forming a cuff 232 about the catheter as shown in FIG. 4. It will be appreciated there are a number of different cuffs, including, for example, but not limited to, those attached to the exterior of the catheter 210 as shown at 232 in FIGS. 3, 4, 5 and 6, those cuffs attached to the interior of the catheter 210 (e.g. stiff tip 230 or distal end 212) as shown at 234 in FIGS. 6 and 6A, or those which are attached to the distal end of the stiff tip 230 or distal end 212 of the catheter 210 over which the balloon is turned back over (i.e. inverted) such as those shown and discussed in more detail in commonly assigned and co-pending U.S. patent application Ser. No. ______, entitled “CATHETER HAVING A BALLOON MEMBER INVERTEDLY ATTACHED THERETO”, filed in the names of Letson et al. on Nov. 30, 2002 (Attorney Docket No.18,477), the disclosure of which is herein incorporated by reference in its entirety. Furthermore, as discussed in more detail in commonly assigned and co-pending U.S. patent application Ser. No. ______, entitled “CATHETER WITH UNITARY COMPONENT”, filed in the names of McMichael et al. on Nov. 30, 2002 (Attorney Docket No.17,110A), the disclosure of which is herein incorporated by reference in its entirety, the catheter 210 may include a balloon 218 which is molded or otherwise attached to or integrally formed with or to the distal end 212 of the catheter 210.

Alternatively, as noted above, the balloon distal end 222 may be attached directly to the end of the shaft 226 or tip 230. In such an alternate embodiment (not shown), no distal cuff will exist, yet the distal end 212 of the catheter 210, which may include a stiff tip 230, will be covered or isolated from contact with the patient by the balloon 218.

It is also contemplated in still another alternate embodiment that the balloon 218 may be attached to the catheter 210 in such a way that it does not cover the distal end 212 of the catheter 210, but may extend beyond or protrude beyond the distal end 212 of the catheter 210 when inflated, so as to keep the distal end 212 of the catheter 210 from coming in contact with and irritating the adjacent tissue when the balloon 218 is inflated.

While the balloon 218 may be attached to the catheter in a variety of manners, the novel balloon orientation may allow for the use of a shorter catheter shaft 226 and thus may decrease or minimize the irritation of anatomical structures associated with prior art devices. That is, more specifically, for example, in gastrostomy tubes or the like, the minimum length of the catheter shaft 226 needed may be reduced as the inflated balloon 218 need not have as large of length dimension as with prior catheters to achieve the necessary and/or desired level of retention force within a patient. That is, more specifically, the catheter shaft 226 does not need to be as long or extend into the cavity of the patient as far, because a balloon 218 of the present invention can achieve the necessary and/or desired level of retention force with less length (as a result of the novel balloon orientation of the present invention). As such, where the balloon 218 length dimension is smaller or shorter than that of prior catheters, the distal end 212 or tip 230 (depending on the embodiment) of the catheter 210 of the present invention may be less likely to come in contact with the opposite side of the body cavity into which the catheter 210 is placed, and therefore is less likely to cause the irritation and/or discomfort associated with such contact. As such the want for a distally extending or protruding balloon may, in some instances, be eliminated, although the use of a distally extending or protruding balloon is generally desirable.

It will be appreciated that catheters having longer tubes, such as, for example, a transjejunal or gastrojejunal tube (not shown), where the distal tip of the catheter generally extends well beyond the balloon and into the one or more cavities (e.g. small intestine or colon) of a patient, the use of a low profile balloon may not enable the shortening or reduction in catheter length where a minimum shaft length is necessary to achieve a desired function or result. The low profile balloon can however, provide one or more other benefits. For example, the low profile balloon may also be a low volume balloon in that it may also take up less space within the patient, thereby providing additional potential benefits. That is, in some embodiments, depending on the size of the catheter and balloon used, the balloon may have a smaller volume as compared to the balloons of the prior art (e.g. those which are more spherical). One will recognize that a balloon with a smaller volume takes up less space within a cavity in a patient's body. As such there is more room for other items, such as nutritional fluids and the like. Accordingly, the use of a low profile and/or low volume balloon can provide more room in a patient's stomach and thus allow for the introduction of more fluids into the stomach, thereby allowing for shorter and/or less frequent feedings. Additionally, the balloon orientation of the present invention is intended to increase the surface area over which the retention force or pressure is applied to the patient. That is, generally, as the width or radial dimension of the balloon increases, so too will the area of the balloon which is readily able to contact the patient, thereby resulting in a greater area over which forces are applied to the patient. Thus, should the head of the catheter be pulled in an outward direction (i.e. away from the patient) while the balloon is inflated the resulting force applied by the balloon against the patient is translated over a greater area of the patient and is desirably less noticeable to the patient.

Having discussed the catheters of the present invention in general terms as well as some of the advantages thereof, the discussion hereof now shifts to a more detailed discussion of the low profile balloon 218 (FIGS. 3, 4, 5, 6, and 6A). Turning to FIG. 6, there is shown a cross-sectional view of the balloon catheter 210 of FIG. 5. As before, the balloon 218 is shown having a width dimension extending from point D to point D′. The reader of the present disclosure will note that the line along which the width dimension (D-D′) of the balloon 218 is taken includes the width dimension of the catheter 210, which is shown as E-E′ (FIG. 6). Unless reference is specifically made to an uninflated embodiment, it will be appreciated that the terms “width” or “width dimension” are intended to mean the inflated width or width dimension of the balloon 218 as measured between points D-D′. Similarly, unless reference is specifically made to an uninflated embodiment, it will also be appreciated that the terms “length” or “length dimension” are intended to mean the inflated length or length dimension of the balloon 218 as measured between points C-C′ (FIG. 5). Although believed to be clear as discussed above, the balloon 218 may have an expandable region as well as one or more cuffs or attachments, such as those indicated at 232 and 234 in FIG. 6. In determining the length dimension or width dimension of the balloon 218 only the portion of the balloon which inflates is to be considered. That is, for example, as the cuffs or attachments 232 and 234 do not inflate they are not to be included when determining the dimensions—as shown in FIGS. 5, 6 and 6A. It is of note that in at least one embodiment the balloon 218 may be constructed or attached to the catheter 210 such that inflation of the balloon 218 results in the balloon 218 extending beyond distal end 212 of the catheter 210 thereby providing an inflated length dimension greater than the uninflated length of the balloon 218.

As shown in FIG. 5, the width dimension (D-D′) of the balloon 218 may be about 1.3 times greater than the length dimension (C-C′) of the inflated balloon 218. It is appreciated that while the balloon 218 of FIG. 5 is shown having an inflated width dimension (D-D′) about 1.3 times greater than its inflated length dimension (C-C′), the size and shape of the balloon 218 may vary with the size and/or the intended use of the catheter 210. That is, it may be desirable for the inflated width dimension (D-D′) of the balloon to be at least about 1.3, more desirably at least about 1.4, even more desirably at least about 1.45, and most desirably at least about 1.5, times larger than the length dimension (C-C′) of the inflated balloon 218, but the ratio may differ depending upon the size and/or use of the catheter 210 as well as the location of the balloon 218 along the catheter 210.

It will be appreciated that the higher the ratio the more the balloon 218 becomes distorted from the circular or spherical shape of the balloons of the prior devices. In fact, the greater the ratio, the more the balloon 218 of the present invention begins to approach or approximate the shape frequently associated with a donut or tire, thereby further differentiating it from the circular shapes of the balloons found in prior catheters.

The balloon 218 described in the present invention may desirably be made of an elastomeric material such as, for example, silicone, although any suitable material may be used. It is also contemplated that the material used to make or form the balloon 218 may not have a uniform thickness throughout. That is, the material forming the balloon 218 may be thicker in one or more regions of the balloon 218 than in other regions of the balloon so as to promote and/or to resist or limit inflation of the balloon 218 in one or more direction. More specifically, for example, the material of the balloon 218 may be formed such that it promotes radial expansion upon inflation so as to enable the desired inflated width dimension to length dimension ratios discussed herein.

The use of non-uniformly thick material or other suitable expansion limiters (such as, for example, the biasing means or annular restraints mentioned above) in conjunction with the present invention may allow the desired specifications of inflated width dimension to inflated length dimension ratios to be achieved with less fluid volume in the balloon than might otherwise be necessary with prior devices.

It will be appreciated that the balloon 218 may be pre-formed in such a manner as to assist in the inflation or otherwise obtain the desired dimensions of an inflated balloon 218. That is, the balloon 218 may be molded or shaped to promote or control width dimension inflation of the balloon relative to the inflation of the length dimension of the balloon 218 or vice versa. Even where pre-formed balloons 218 are not used, a balloon material which has high-strength and/or high stress properties is desired. That is, it is desirable for the material used to form a balloon 218 to have a high tensile strength (about 1200 to about 1400 psi), a high tear strength (about 140 to about 200 ppi), be capable of exposure to significant elongation (about 600 to about 800%) and have a soft feel (about 25 to about 30 shore “A” durometer). The combination of a pre-formed balloon and the use of high-strength materials can assist in the achievement of the desired balloon ratios. The use of a pre-shaped or pre-formed balloon may enable the balloons 218 of the catheters 210 to achieve desired sizes and/or shapes which heretofore were not achievable.

It is of note that the volume of the balloon 218 need not have an established minimum volume level so long as the retention force (i.e. the force necessary to maintain the catheter in place within the patient) is achieved and maintained. That is, it will be appreciated that the volume of fluid needed to achieve a retention force may vary depending on the size and shape of the balloon as well as the patient and the catheter it is being used with.

It will be further appreciated that the balloon may have various degrees of inflation. That is, once inflation of the balloon begins and until the balloon reaches at least a significant inflation, the balloon is at least partially inflated and will have an inflated width dimension and a length dimension, albeit only a portion of the at least significantly inflated or fully inflated dimensions. As such, all references herein to measurements of a balloon, unless stated to the contrary, are measurements of a balloon which is at a minimum at least significantly inflated.

It will also be appreciated that reference to an at least significantly inflated balloon refers to a balloon which has been inflated so as to provide the minimum designed and/or desired retention force. Stated another way, this value may, for example, be referred to as the manufacturer's recommended inflation value, the manufacturer's minimum recommended inflation value or the like. It is contemplated that a range of volumes may exist in which the balloon may be considered at least significantly inflated. As further clarification, a balloon may be considered at least significantly inflated once a volume of fluid has been provided therein which is sufficient to provide the minimum retention force to retain the catheter within the patient as desired.

Once a balloon has achieved at least a significant level of inflation, it shall be considered to be inflated until it reaches a level of overinflation. For purposes of this disclosure, a balloon shall be considered to have reached a level of overinflation or to be overinflated when, as a result of inflation, the balloon experiences significant permanent deformation or when the life of the balloon is adversely affected as a result of inflation forces applied thereto. In many embodiments, overinflation occurs when the manufacturer's recommended maximum inflation level is exceeded. Thus, it will be appreciated that actual inflation can exceed the inflated level, however, it is generally not recommended to do so.

Nevertheless, the present application is intended to cover all such states of the balloon (e.g., but not limited to, partially inflated, underinflated, at least significantly inflated, fully inflated, overinflated, etc.) provided that when the balloon is at least significantly inflated the ratios of the measurements as discussed herein are satisfied.

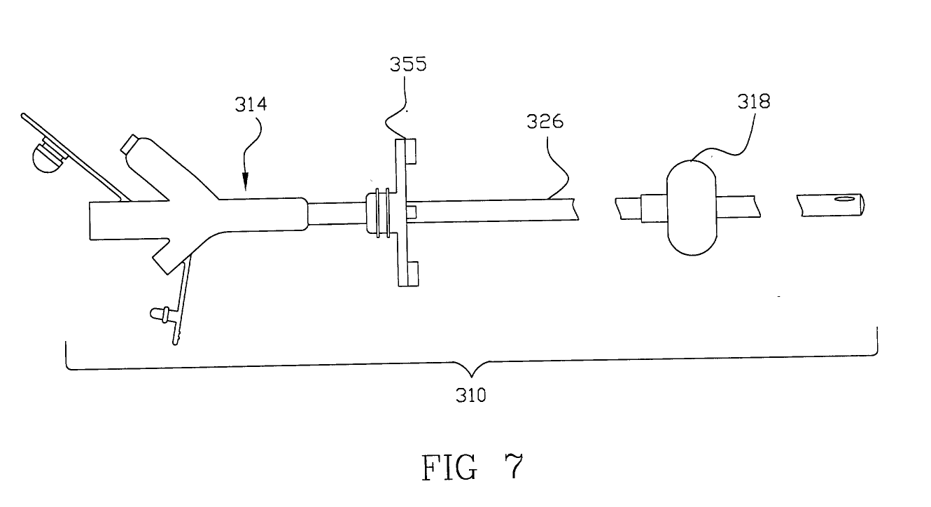

As shown in FIG. 7, the balloon catheter 310 of the present invention may have a large head or a non-low profile head 314. In those embodiments of the present invention having the traditional or non-low profile head 314, the catheter 310 may further include an adjustable ring 355 or the like slidably mounted along the catheter 310, and medially thereof, and sized to frictionally engage the catheter shaft 326. The securement of the ring 355 to the catheter shaft 326 is provided solely by frictional engagement therebetween. The locking ring 355, desirably made of silicone material or the like, is manually adjustable along the catheter shaft 326 to accommodate to the size of the patient (not shown). The balloon 318 and the ring 355 both work to maintain the positioning of the catheter 310 within the patient. When desired, the ring 355 may be retracted along the catheter shaft 326 away from the patient's body (not shown) by merely overcoming the frictional engagement forces between the ring 355 and catheter shaft 326. Any suitable adjustable ring 355 or retention device will work, however, one example of such a ring is a SECUR-LOK* ring available from Ballard Medical Products (having offices in Utah), a subsidiary of Kimberly-Clark Corporation.

In sum, each of the different embodiments of the present invention, as representatively illustrated and described herein, advantageously provide an improved catheter which may exhibit one or more of the following: (1) an improved seal between the catheter and the patient, (2) less leakage of stomach contents, (3) an increase in the surface area contact between the balloon and the patient, (4) a reduction in the pressure exerted on the catheter shaft by the balloon thereby reducing constriction on the shaft and allowing for increased or less restricted flow therethrough, (5) a reduction in the amount of the patient's body cavity taken up by the balloon, (6) use of a shorter catheter shaft, thereby allowing for a reduction in the incidence of irritating contact between the distal end of the catheter and adjacent anatomical surfaces, (7) less irritation of the gastric wall, (8) the dispersion of pressure exerted by the balloon on the patient (e.g., stomach or

1. A catheter having an interior and an exterior, the catheter comprising:
a means for expansion having a proximal end, a distal end, a length dimension and a width dimension;
a shaft including a distal end, a first lumen configured for fluid communication with a body cavity, and a second lumen configured for communication with the means for expansion; and
wherein the width dimension of the means for expansion is greater than the length dimension of the means for expansion when the means for expansion is expanded.

2. The catheter of claim 1, wherein the means for expansion has a width dimension to length dimension ratio of at least about 1.3 to 1 when the means for expansion is expanded.

3. The catheter of claim 1, wherein the means for expansion has a width dimension to length dimension ratio of at least about 1.4 to 1 when the means for expansion is expanded.

4. The catheter of claim 1, wherein the means for expansion has a width dimension to length dimension ratio of at least about 1.5 to 1 when the means for expansion is expanded.

5. The catheter of claim 1 wherein the means for expansion is a balloon, an expandable sleeve, or an inflatable member.

6. The catheter of claim 1, wherein the catheter further comprises a tip attached to the distal end of the shaft, the tip having a proximal end, a distal end, and an interior defining a passageway; and
wherein the proximal end of the means for expansion is attached to the shaft and the distal end of the means for expansion is attached to the interior of the tip.

7. The catheter of claim 1, wherein the catheter further comprises a tip attached to the shaft, the tip having a proximal end, a distal end, and an interior defining a passageway; and
wherein the proximal end of the means for expansion is attached to the shaft and the distal end of the means for expansion is integrally formed with the tip.

8. The catheter of claim 6, wherein the means for expansion extends distally from the distal end of the tip, and then circumscribes the distal end of the tip.

9. The catheter of claim 7, wherein the means for expansion extends distally from the distal end of the catheter, and then circumscribes the distal end of the catheter.

10. The catheter of claim 1, wherein the means for expansion comprises an elastomeric material.

11. A catheter comprising:
a head having at least two openings through which fluid may be passed;
a catheter shaft extending from the head, the catheter shaft having a first and second lumen, each of the lumens being in communication with at least one of the two openings, the catheter shaft further having an interior and an exterior; and
a means for expansion having a first end attached to the catheter shaft and a second end attached to the catheter shaft, wherein the means for expansion has a width dimension at least about 1.4 times greater than the length dimension of the means for expansion when the means for expansion is expanded.

12. The catheter of claim 11, wherein the first end of the means for expansion is attached to the exterior of the catheter shaft, and the second end of the means for expansion is attached to the interior of the catheter shaft.

13. The catheter of claim 11, wherein the first end of the means for expansion is attached to the exterior of the catheter shaft, and the second end of the means for expansion is attached to the exterior of the catheter shaft.

14. The catheter of claim 11, wherein the catheter shaft further comprises
a distal end; and
a tip attached to the distal end of the catheter shaft.

15. The catheter of claim 14, wherein the first end of the means for expansion is attached to the exterior of the catheter shaft, and wherein the second end of the means for expansion is attached to the interior of the tip.

16. The catheter of claim 11, wherein the head has a plurality of valves configured for selectively controlling fluid flow through the catheter.

17. A catheter comprising:
an elongate shaft having at least one lumen extending longitudinally therethrough, a distal end and a width dimension; and
an expandable balloon, said balloon having a length dimension and a width dimension;
wherein the width dimension of the balloon is larger than the length dimension of the balloon when the balloon is expanded.

18. The catheter of claim 17, wherein the width dimension of the balloon is at least about 1.4 times larger than the length dimension of the balloon when the balloon is expanded.

19. The catheter of claim 17, wherein the width dimension of the balloon is at least about 1.45 times larger than the length dimension of the balloon when the balloon is expanded.

20. The catheter of claim 17, wherein the width dimension of the balloon is at least about 1.5 times larger than the length dimension of the balloon when the balloon is expanded.

21. The catheter of claim 17, wherein the shaft further comprises
a distal end; and
a tip attached to the distal end of the shaft.

22. The catheter of claim 21 further comprising a head; wherein the balloon is located between the head and the distal tip of the catheter.

23. The catheter of claim 17, wherein the balloon has at least one cuff, and wherein one of the cuffs is located at or near the distal end of the catheter.

24. The catheter of claim 17, wherein the balloon has at least one cuff, and wherein at least one of the cuffs is attached to the interior of the catheter.

25. A balloon catheter configured for placement through a stoma into a body cavity so that the catheter is maintained in the stoma, the catheter comprising:
a head having at least one opening through which a fluid may be introduced;
a catheter shaft extending from the head to a distal end the catheter shaft forming a passageway, said passageway having an interior; and
an expandable member attached to the catheter shaft about the passageway so as to form a balloon, the balloon being in collapsed state when not expanded; wherein the balloon has a first end, a second end, a length dimension and a width dimension; and
wherein the balloon extends radially outwardly from the catheter shaft when expanded, such that the width of the balloon is at least about 1.3 times the length of the balloon when expanded.

26. The balloon catheter of claim 25, wherein a first end of the balloon is attached to the catheter shaft so as to form a proximal cuff of the balloon, and wherein a second end of the balloon is attached to the catheter shaft so as to form a distal cuff of the balloon.

27. The balloon catheter of claim 25, wherein the distal end of the balloon is attached to the distal end of the catheter and wherein the distal end of the balloon defines a portion of the passageway through the catheter shaft.

28. The balloon catheter of claim 25, wherein the catheter shaft comprises a tip attached to the distal end of the catheter shaft, opposite the head.

29. The balloon catheter of claim 28, wherein the balloon is attached to the exterior of the catheter shaft and the interior of the tip.
